(19) United States
(12) Patent Application Publication
(10) Pub. No.: US 2026/0199199 A1
(43) Pub. Date: Jul. 16, 2026

(54) COMPOSITION COMPRISING AT LEAST ONE SATURATED FATTY ALCOHOL, AT LEAST ONE ANIONIC SURFACTANT, AT LEAST ONE POLYOL AND AT LEAST ONE NON-SILICONE OIL, THE COMPOSITION BEING FREE OF SILICONE COMPOUNDS

(71) Applicant: L'OREAL, PARIS (FR)

(72) Inventors: Anne FALIP, CHEVILLY-LARUE (FR); Majolie LIBII, CHEVILLY-LARUE (FR); Carole GUIRAMAND, CHEVILLY-LARUE (FR)

(21) Appl. No.: 19/138,001

(22) PCT Filed: Dec. 14, 2023
(86) PCT No.: PCT/EP2023/085801

§ 371 Date: Jun. 11, 2025

(30) Foreign Application Priority Data
Dec. 15, 2022 (FR) FR2213440

(51) Int. Cl. A61K 8/06; A61K 8/34; A61K 8/44; A61K 8/46; A61K 8/92; A61Q 5/00; A61Q 19/08

(52) U.S. Cl. CPC A61K 8/062; A61K 8/34; A61K 8/44; A61K 8/466; A61K 8/92; A61Q 5/00; A61Q 19/08; A61K 2800/34

(57) ABSTRACT

The present invention relates to a composition, preferably a cosmetic composition, in the form of an oil-in-water emulsion, in which an oily phase is dispersed in an aqueous phase, comprising at least: (a) a saturated C16-C22 fatty alcohol; (b) an anionic surfactant including at least one C16-C22 hydrocarbon-based chain; (c) a polyol present in an amount strictly greater than 15% by weight relative to the total weight of the composition, and (d) at least one non-silicone oil present in an amount of greater than or equal to 8% by weight relative to the total weight of the composition, the composition being free of silicone compounds. The invention also relates to a cosmetic treatment process in which said composition is applied to a keratin material, and to the use of said composition in the cosmetic field, in particular for caring for, protecting and making up the skin, or for haircare.

### TECHNICAL FIELD

The present invention relates to a composition, preferably a cosmetic composition, in the form of an oil-in-water emulsion, in which an oily phase is dispersed in an aqueous phase, comprising:
(a) at least one saturated C16-C22 and preferably C16-C18 fatty alcohol;
(b) at least one anionic surfactant including at least one C16-C22 and preferably C16-C18 hydrocarbon-based chain;
(c) at least one polyol present in an amount strictly greater than 15% by weight relative to the total weight of the composition, and
(d) at least one non-silicone oil present in an amount of greater than or equal to 8% by weight relative to the total weight of the composition, the composition being free of silicone compounds, and to the use of said composition in the cosmetic and dermatological fields, in particular for caring for and treating keratin materials.

### PRIOR ART

The particular type of galenic form targeted by the present invention, namely an oil-in-water emulsion notably requiring the presence of at least one saturated C16-C22 fatty alcohol and at least one anionic surfactant including at least one C16-C22 hydrocarbon-based chain, allows active agents to be conveyed while at the same time affording advantageous sensory properties that are appreciated by users when it is applied to keratin materials.

Oil-in-water emulsion compositions notably requiring the presence of at least one saturated C16-C22 fatty alcohol and at least one anionic surfactant including at least one C16-C22 hydrocarbon-based chain are described in particular in WO 2018/108878 and WO 2020/002538.

However, these compositions address very specific objectives. Their purpose is notably, respectively, to mask skin imperfections, moisturize the skin and/or treat the signs of skin ageing, in particular the appearance of fine lines and deep wrinkles, particularly as they increase with age.

The term “keratin material” notably means the skin, the scalp, keratin fibres such as the eyelashes, the eyebrows, head hair, bodily hair, the nails, and mucous membranes such as the lips, and more particularly the skin (body, face, neck, area around the eyes, eyelids).

However, it is sometimes difficult to reconcile the requirements in terms of sensory properties, texture and stability. In particular, it is desirable to have available compositions that are capable of maintaining the same viscosity over time, in particular a viscosity which neither increases nor decreases over time.

### DISCLOSURE OF THE INVENTION

The aim of the present invention is to solve the abovementioned technical problems.

In the light of the foregoing, it is thus clear that there is still a need for compositions with advantageous sensory properties, and in particular good glidance and a non-tacky aspect, and a reduction or absence of soaping, while at the same time retaining satisfactory stability, in particular stability evaluated at 1 month and/or 2 months of storage, in particular macroscopically and microscopically after two months of storage under temperature conditions ranging from 4° C. to 45° C., in particular at 4° C., room temperature (generally between 20° C. and 25° C.), and 45° C.

Finally, consumers are nowadays very sensitive with regard to the environmental impact of the products used.

Consequently, it remains very difficult to obtain cosmetic compositions which offer particular sensory properties in terms of a glidant and non-tacky feel on application, with little soaping, while at the same time retaining good stability over time.

### SUMMARY OF THE INVENTION

Thus, according to a first aspect, the present invention relates to a composition, preferably a cosmetic composition, in the form of an oil-in-water emulsion, in which an oily phase is dispersed in an aqueous phase, comprising:
(a) at least one saturated C16-C22 and preferably C16-C18 fatty alcohol;
(b) at least one anionic surfactant including at least one C16-C22 and preferably C16-C18 hydrocarbon-based chain;
(c) at least one polyol present in an amount strictly greater than 15% by weight relative to the total weight of the composition, and
(d) at least one non-silicone oil present in an amount of greater than or equal to 8% by weight relative to the total weight of the composition,
the composition being free of silicone compounds.

For the purposes of the present invention, the term “free of silicone compounds” refers to a composition according to the invention which comprises less than 2% by weight, preferably less than 1% by weight, relative to the total weight of the composition, and more preferentially is free (devoid) of silicone compounds.

The term “silicone compounds” (also called silicones) means any silicone compounds that are liable to be used in the cosmetic and/or dermatological fields, i.e. inorganic compounds consisting at least of a mixture of silicon and oxygen, notably including silicone oils, silicone surfactants, dimethylsiloxane polymers, volatile or functionalized silicones, silicone elastomers, silicone resins, phenyl silicones or amino silicones.

As illustrated in the examples hereinbelow, the Applicant has discovered, surprisingly, that it is possible to obtain compositions with advantageous sensory properties, and in particular good glidance and a non-tacky aspect, with little soaping, while at the same time retaining satisfactory stability over time.

The compositions according to the present invention advantageously enable ingredients of various kinds such as cosmetic active agents, sunscreens, particles, polymers, dyes, nacres or pigments and fragrances to be conveyed, depending on the various benefit(s) desired.

The compositions according to the present invention also have the advantage of affording an anti-ageing effect.

According to a particular embodiment, the present invention concerns a composition, notably a cosmetic composition, in the form of an oil-in-water emulsion, in which an oily phase is dispersed in an aqueous phase, comprising:
(a) at least one saturated C16-C22 and preferably C16-C18 fatty alcohol;
(b) at least one anionic surfactant including at least one C16-C22 and preferably C16-C18 hydrocarbon-based chain;
(c) at least one polyol present in an amount strictly greater than 15% by weight relative to the total weight of the composition, and
(d) at least one non-silicone oil present in an amount of greater than or equal to 8% by weight relative to the total weight of the composition,
the composition being free of silicone compounds,
in which the anionic surfactant(s) including at least one C16-C22 hydrocarbon-based chain are chosen from the surfactants of general formula (I): RCOY(CH₂)ₙSO₃M in which R represents a saturated, linear or branched C16-C22 alkyl group; Y represents —O— or —NR₁— with R₁ representing a linear or branched C1-C3 alkyl group; M is chosen from the group formed by hydrogen, alkali metals, alkaline-earth metals, the ammonium group and organic amines; n is an integer ranging from 1 to 3.

The invention also relates to a process for the cosmetic treatment of a keratin material in which a composition as defined previously is applied to the keratin material.

The present invention also relates to the use of a composition as described previously in the cosmetic field, and in particular for caring for, protecting and/or making up the skin of the body or face, or for haircare.

Other characteristics, aspects and advantages of the invention will become apparent on reading the detailed description that follows.

### DETAILED DESCRIPTION

The composition according to the invention is cosmetic and/or dermatological, and preferably is cosmetic.

The composition according to the invention is generally suitable for topical application to the skin and thus generally comprises a physiologically acceptable medium, i.e. a medium that is compatible with the skin.

The term “cosmetic” refers to a composition that is compatible with the skin, mucous membranes and the integuments. The composition according to the invention is non-therapeutic.

It is preferably a cosmetically acceptable medium, i.e. a medium which has a pleasant colour, odour and feel and which does not cause any unacceptable discomfort, i.e. stinging, tautness or redness, liable to discourage the user from applying this composition.

In the text hereinbelow, the expression “at least one” is equivalent to “one or more” and, unless otherwise indicated, the limits of a range of values are included in that range.

### Saturated C16-C22 Fatty Alcohol

The term “saturated fatty alcohol” means any alcohol comprising a linear saturated (containing no covalent double or triple bonds) hydrocarbon-based chain, in particular a linear alkyl radical, said chain comprising between 16 and 22 carbon atoms and a hydroxyl function.

The term “hydrocarbon-based chain” means an organic group predominantly consisting of hydrogen atoms and carbon atoms.

The saturated fatty alcohol(s) present in the composition in accordance with the invention comprise between 16 and 22 carbon atoms, preferably between 16 and 18 carbon atoms. The saturated C16-C22 fatty alcohol(s) used in the compositions in accordance with the present invention are solid at room temperature and advantageously bear a chain-end—OH group.

The saturated fatty alcohol(s) that are of use in the context of the present invention may notably be chosen from cetyl alcohol or hexadecanol (C₁₆), stearyl alcohol or octadecanol (Cis), or behenyl alcohol (C₂₂) which are solid at room temperature and advantageously bear a chain-end —OH group.

Particularly preferably, use will be made of one or more alcohols chosen from cetyl alcohol, stearyl alcohol, and mixtures thereof such as cetylstearyl alcohol.

Preferably, the composition according to the invention comprises a mixture of C₁₆ and C₁₈ fatty alcohols; in particular, the composition comprises cetylstearyl alcohol.

More preferably, the mixture of C₁₆ and C₁₈ fatty alcohols is used in a C₁₆/C₁₈ mass ratio ranging from 20/80 to 80/20 and advantageously in a C₁₆/C₁₈ mass ratio equal to 50/50.

The amount of fatty alcohols in the composition in accordance with the invention is between 2.5% and 10% by weight, preferably between 2.5% and 8% by weight and even more preferentially between 2.7% and 7% by weight, relative to the total weight of the composition.

### Anionic Surfactants Including at Least One C16-C22 Hydrocarbon-Based Chain

The anionic surfactant(s) that are useful in the context of the invention comprise at least one saturated (not containing any covalent double or triple bonds) or unsaturated (which may contain one or more covalent double and/or triple bonds), preferably saturated, linear hydrocarbon-based chain, in particular a linear alkyl radical, said chain comprising from 16 to 22 carbon atoms, preferably from 16 to 18 carbon atoms. Preferably, the anionic surfactant(s) that are useful in the context of the invention comprise only one saturated or unsaturated linear hydrocarbon-based chain.

According to a particular embodiment, the anionic surfactant(s) that are useful in the context of the invention contain only one C16-C22 hydrocarbon-based chain.

The term “hydrocarbon-based chain” means an organic group predominantly consisting of hydrogen atoms and carbon atoms.

According to a particular embodiment of the invention, the anionic surfactant(s) including at least one C16-C22 hydrocarbon-based chain are chosen from surfactants comprising at least one sulfonate function, acylglutamates, and mixtures thereof.

According to a preferred embodiment of the invention, the anionic surfactant(s) comprising at least one sulfonate function are chosen from (C16-C22)alkylsulfonates, (C16-C22)alkylamidesulfonates, (C16-C22)alkylarylsulfonates, (C16-C22)alkylsulfoacetates, N-acyl(C16-C22)-N-(C1-C6)alkyltaurates, (C16-C22)acylisethionates, (C16-C22)alkylsulfolaurates, and mixtures thereof.

Preferentially, the anionic surfactant(s) comprising at least one sulfonate function are chosen from:
acylisethionates, the linear or branched acyl group comprising from 16 to 22 carbon atoms, preferably from 16 to 18 carbon atoms;
N-acyl-N-alkyltaurates, the linear or branched acyl group comprising from 16 to 22 carbon atoms, preferably from 16 to 18 carbon atoms, and the linear or branched alkyl group comprising from 1 to 6 carbon atoms, or cyclic group comprising from 3 to 6 carbon atoms; preferably, the alkyl group is a methyl,
and mixtures thereof;
   in particular in the form of alkali metal or alkaline-earth metal, ammonium or amino alcohol salts.

As N-acyl-N-alkyltaurates, mention may be made of sodium palmitoyl methyltaurate sold under the name Nikkol PMT® by the company Nikkol; the sodium salt of N-stearoyl N-methyl taurate sold under the name Nikkol SMT by the company Nikko.

The acyl glutamate(s) may be chosen from acyl glutamates in which the acyl group comprises from 16 to 18 carbon atoms.

Examples of acyl glutamates that may notably be mentioned include palmitoyl glutamic acid, stearoyl glutamic acid, behenoyl glutamic acid, olivoyl glutamic acid, and the salts of these acids, notably the alkali metal salts such as the Na, Li or K and preferably Na or K salts, the alkaline-earth metal salts such as the Mg salts or the ammonium salts of said acids.

Mention may be made, for example, of the compounds having the INCI name sodium stearoyl glutamate, sodium olivoyl glutamate, and mixtures thereof.

As acylglutamic acid salts, mention may also be made of sodium stearoyl glutamate, such as the product sold under the reference Acylglutamate HS 11 by the company Ajinomoto and disodium hydrogenated stearoyl glutamate, such as the product sold under the reference Acylglutamate HS-21 by the company Ajinomoto.

According to a particular embodiment of the invention, the anionic surfactant(s) including at least one C16-C22 hydrocarbon-based chain are chosen from the surfactants of general formula (I): RCOY(CH₂)ₙSO₃M in which R represents a saturated, linear or branched C16-C22 alkyl group; Y represents —O— or —NR₁— with R₁ representing a linear or branched C1-C3 alkyl group; M is chosen from the group formed by hydrogen, alkali metals, alkaline-earth metals, the ammonium group and organic amines; n is an integer ranging from 1 to 3. Preferably, the anionic surfactant of general formula (I) is N-stearoyl-N-methyltaurate, with M corresponding to Na.

The composition according to the invention advantageously comprises from 0.05% to 5%, preferably from 0.1% to 2% and even more preferentially from 0.1% to 1.5% by weight, relative to the total weight of the composition, of one or more anionic surfactants including at least one C16-C22 hydrocarbon-based chain as defined previously.

In the compositions in accordance with the invention, the fatty alcohols (a)/anionic surfactants (b) mass ratio may be between 10:1 and 6:4, preferably between 19:2 and 7:3, and even more preferentially between 9:1 and 4:1.

Without wishing to be bound by any theory, it appears that the fatty alcohol(s) and the anionic surfactant(s) including at least one C16-C22 hydrocarbon-based chain form, with the hydrophilic phase, an alpha crystalline phase, also referred to as alpha gel. This phase may notably be characterized by DSC analysis and by X-ray diffraction at a temperature lower than the melting point of the alpha gel phase.

Thus, the composition in accordance with the invention is capable of forming crystalline lamellar phases.

According to a particular embodiment of the invention, the compositions of the invention contain at least one swollen alpha-crystalline phase having a swelling period of greater than 10 nm, preferably greater than 12 nm and even more preferentially greater than or equal to 15 nm. The period is defined as being the sum of the thickness of a bilayer formed by the fatty alcohols (a) and the anionic surfactants (b) and the thickness of the water layer between two leaflets, this unit being repeated several times.

According to a particular embodiment, in a composition according to the invention, the difference between the number of carbon atoms in the hydrocarbon-based chain of the anionic surfactant(s) (b) and the number of carbon atoms in the fatty alcohol(s) (a) must not differ by more than 5 carbon atoms, and is preferentially less than or equal to 4.

### Polyols

The composition according to the invention comprises at least one polyol in a content strictly greater than 15% by weight relative to the total weight of the composition.

Polyols are defined as being organic molecules comprising at least two hydroxyl (OH) functions.

For the purposes of the invention, the term “polyol” means:
a saturated or unsaturated, linear or branched hydrocarbon-based chain comprising at least two hydroxyl functions; or
a saturated, linear or branched hydrocarbon-based chain in which one or more carbon atoms are replaced with an oxygen atom and which comprises at least two hydroxyl functions, for instance polyethylene glycols (PEGs) containing from 4 to 8 ethylene glycol units.

Preferably, the polyol(s) bear a saturated, linear or branched hydrocarbon-based chain. Advantageously, the polyol(s) comprise a number of carbon atoms ranging from 2 to 20, preferably from 2 to 10, and comprise from 2 to 12 and better still from 2 to 8 hydroxyl functions.

The polyol(s) may be chosen from ethylene glycol, propylene glycol, 1,3-propanediol, isoprene glycol, butylene glycol, dipropylene glycol, polypropylene glycol, caprylyl glycol, glycerol, diglycerol, erythritol, pentaerythritol, arabitol, adonitol, sorbitol, dulcitol, maltitol and panthenol.

Among these polyols, glycerol, caprylyl glycol, propylene glycol, dipropylene glycol, butylene glycol, propane-1,3-diol, and mixtures thereof, are preferentially chosen.

According to one embodiment, the polyol(s) comprise from 2 to 12 hydroxyl functions, preferably from 2 to 8 hydroxyl functions, and in particular are chosen from ethylene glycol, propylene glycol, propane-1,3-diol, isoprene glycol, butylene glycol, dipropylene glycol, polypropylene glycol, caprylyl glycol, glycerol, diglycerol, erythritol, pentaerythritol, arabitol, adonitol, sorbitol, dulcitol, maltitol, panthenol and mixtures thereof, preferably glycerol, caprylyl glycol, propylene glycol, dipropylene glycol, butylene glycol, propane-1,3-diol and mixtures thereof, and even more preferentially glycerol, caprylyl glycol and mixtures thereof.

Preferably, the polyol is chosen from glycerol, caprylyl glycol and mixtures thereof.

According to another particular embodiment of the invention, the polyol(s) may be present in the composition in an amount of between 15.1% and 50% by weight, preferably between 18% and 45% by weight, even more preferentially between 19% and 40% by weight and better still between 20% and 35% by weight relative to the total weight of the composition.

### Non-Silicone Oils

The composition according to the invention comprises at least one non-silicone oil in a content of greater than or equal to 8% by weight relative to the total weight of the composition.

The term “oil” means a water-immiscible non-aqueous compound that is liquid at room temperature (20° C.) and at atmospheric pressure (760 mmHg).

The composition according to the invention may in particular comprise hydrocarbon-based oils.

They may be of animal, plant, mineral or synthetic origin.

For the purposes of the present invention, the term “hydrocarbon-based oil” means an oil mainly containing hydrogen and carbon atoms.

The term “non-silicone oil” means an oil not comprising any silicon atoms and notably no Si—O groups.

The oils may optionally comprise oxygen, nitrogen, sulfur and/or phosphorus atoms, for example in the form of hydroxyl or acid radicals.

Hydrocarbon-based oils that may notably be mentioned include:
branched alkanes comprising more than 8 carbon atoms, notably C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof,
linear alkanes comprising more than 8 carbon atoms, in particular from 10 to 30 carbon atoms, in particular from 10 to 26 carbon atoms, and more particularly from 10 to 20 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155059 from the company Cognis, and mixtures thereof; or a mixture of alkanes of 15 to 19 carbon atoms (called C15-C19 alkane), for example the products sold under the references Emogreen L19 and Emosmart L19 from SEPPIC,
hydrocarbon-based oils of animal origin,
linear or branched hydrocarbons of mineral or synthetic origin, such as petroleum jelly, polydecenes, hydrogenated polyisobutene such as Parleam, and squalane, and mixtures thereof,
hydrocarbon-based oils of plant origin, such as glyceride triesters, which are generally triesters of fatty acids and of glycerol, the fatty acids of which may have varied chain lengths of from 4 to 24 carbon atoms, these chains possibly being linear or branched, and saturated or unsaturated; these oils are notably wheatgerm oil, rice bran oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil, musk rose oil, Limnanthes alba seed oil (INCI name: Limnanthes alba (Meadowfoam) Seed Oil); or caprylic/capric acid triglycerides such as those sold by the company Stdarinerie Dubois or those sold under the names Miglyol 810, 812 and 818 by the company Dynamit Nobel. Mention may also be made of cocoyl caprylate/caprate, for example sold under the name Cetiol LC by the company Cognis or under the name DUB 810 C by the company Stdarinerie Dubois,
synthetic ethers containing from 10 to 40 carbon atoms, such as dicapryl ether,
synthetic esters, such as the oils of formula R₁COOR₂, in which R₁ represents a linear or branched fatty acid residue including from 1 to 40 carbon atoms and R₂ represents a hydrocarbon-based chain that is notably branched, containing from 1 to 40 carbon atoms, with the proviso that R₁+R₂ is greater than or equal to 10, for instance purcellin oil (cetostearyl octanoate), isopropyl myristate, myristyl myristate, isopropyl palmitate, alkyl benzoates containing between 12 and 15 carbon atoms, such as the product sold under the trade name Finsolv TN or Witconol TN by the company Witco or Tegosoft TN by the company Evonik Goldschmidt, 2-ethylphenyl benzoate, such as the commercial product sold under the name X-Tend 226 by the company ISP, isopropyl lanolate, hexyl laurate, diisopropyl adipate, isononyl isononanoate, oleyl erucate or (Z)-octadec-9-enyl (Z)-docos-13-enoate, 2-ethylhexyl palmitate, isostearyl isostearate, diisopropyl sebacate such as the product sold under the name Dub Dis by the company Stdarinerie Dubois, alcohol or polyalcohol octanoates, decanoates or ricinoleates, such as propylene glycol dioctanoate; hydroxylated esters, such as isostearyl lactate, diisostearyl malate; and pentaerythritol esters, in particular pentaerythrityl tetraoctanoate (INCI name: Pentaerythrityl Tetraethylhexanoate); dipentaerythrityl hexacaprylate/hexacaprate, citrates, such as the ester of C₃-C₂₂ tricarboxylic acid and C₁-C₆ alcohols having the INCI name Triethyl Citrate, for example the product sold under the name Citrofol AI Extra by the company Jungbunzlauer; tartrates such as linear dialkyl tartrates containing 12 or 13 carbon atoms, for example those sold under the name Cosmacol ETI by the company Enichem Augusta Industriale, and also linear dialkyl tartrates containing between 14 and 15 carbon atoms, for example those sold under the name Cosmacol ETL by the same company, and acetates,
polyol esters and pentaerythritol esters, for instance dipentaerythrityl tetrahydroxystearate/tetraisostearate,
esters of diol dimer and diacid dimer, where appropriate esterified on their free alcohol or acid function(s) with acid or alcohol radicals, in particular dimer dilinoleate esters; more particularly chosen from the esters having the following INCI nomenclature: bis-behenyl/isostearyl/phytosteryl dimer dilinoleyl dimer dilinoleate (Plandool G), phytosteryl/isostearyl/cetyl/stearyl/behenyl dimer dilinoleate (Plandool H or Plandool S), and mixtures thereof,
fatty amides, such as isopropyl N-lauroyl sarcosinate, for example the product sold under the trade name Eldew SL205 from Ajinomoto,
fatty alcohols that are liquid at room temperature, with a branched and optionally unsaturated carbon-based chain containing from 12 to 26 carbon atoms, for instance octyldodecanol, 2-butyloctanol or 2-undecylpentadecanol,
higher C₁₉-C₂₂ fatty acids;
carbonates, such as dicaprylyl carbonate, for example the product sold under the name Cetiol CC by the company Cognis.

According to a particular embodiment of the invention, the non-silicone oil(s) are chosen from branched or linear alkanes comprising more than 8 carbon atoms, hydrocarbon-based oils of animal origin, linear or branched hydrocarbons of mineral or synthetic origin, hydrocarbon-based oils of plant origin, synthetic ethers including from 10 to 40 carbon atoms, and synthetic esters of formula R₁COOR₂, in which R₁ represents a residue of a linear or branched fatty acid containing from 1 to 40 carbon atoms and R₂ represents a hydrocarbon-based chain, notably a branched chain containing from 1 to 40 carbon atoms, on condition that R₁+R₂ is greater than or equal to 10, polyol esters and pentaerythritol esters, diol dimer and diacid dimer esters, fatty amides, fatty alcohols that are liquid at room temperature, with a branched and optionally unsaturated carbon-based chain containing from 12 to 26 carbon atoms, higher C₁₉-C22 fatty acids, carbonates and mixtures thereof; in particular from oleyl erucate, branched or linear alkanes including more than 8 carbon atoms, and mixtures thereof; more particularly from oleyl erucate, a mixture of alkanes of 15 to 19 carbon atoms, and mixtures thereof.

The composition according to the invention advantageously comprises from 8% to 40% by weight, preferably from 9% to 25% by weight, and even more preferentially from 10% to 15% by weight of non-silicone oil(s) relative to the total weight of the composition.

### Waxes

A composition according to the invention may comprise at least one wax, notably a wax of plant origin with a melting point above 25° C.

For the purposes of the present invention, the term “wax” means a lipophilic compound that is solid at room temperature (25° C.), with a solid/liquid reversible change of state.

For the purposes of the invention, the melting point corresponds to the temperature of the most endothermic peak observed on thermal analysis (differential scanning calorimetry or DSC) as described in the standard ISO 11357-3:1999.

The melting point of the wax may be measured using a differential scanning calorimeter (DSC), for example the calorimeter sold under the name MDSC 2920 by the company TA instruments.

The measuring protocol is as follows: a sample of 5 mg of wax placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

As waxes of plant origin with a melting point above 25° C. that may be used according to the invention, mention may be made of:
carnauba wax,
candelilla wax, in particular the commercial reference Candelilla Wax SP 75 G from the company Strahl & Pitsch,
lanolins and lanolin derivatives such as acetylated lanolins, oxypropylenated lanolins or isopropyl lanolate, and mixtures thereof,
rice bran wax,
ouricury wax,
esparto grass wax,
cork fibre wax,
sugar cane wax,
Japan wax,
sumac wax,
montan wax,
orange wax, in particular the product sold under the reference Orange Peel Wax by the company Koster Keunen,
laurel wax,
sunflower wax,
lemon wax,
olive wax,
berry wax,
beeswax, in particular the product sold under the name White Beeswax SP 453P by the company Strahl & Pitsch or Cerabeil Lor by the company Baerlocher,
buckwheat wax, in particular the product sold by the company Codif,
waxes obtained by hydrogenation of olive oil esterified with C12 to Cis fatty alcohols, in particular those sold by the company Sophim in the Phytowax range (12L44, 14L48, 16L55 and 18L57),
partially hydrogenated wax esters of jojoba oil,
hydrogenated jojoba oil,
isomerized jojoba oil, in particular the trans-isomerized partially hydrogenated jojoba oil manufactured or sold by the company Desert Whale under the trade name Iso-Jojoba-500,
fatty acid esters of polyglycerol, in particular the mixture of three waxes jojoba esters & Helianthus annuus seed wax & Acacia decurrens extract and polyglyceryl, in particular the product sold under the name Hydracire S or Acticire® by the company Gattefossé,
hydrogenated glycerol esters, in particular the product sold under the name Cegesoft HF 52 by Cognis (BASF), a mixture of hydrogenated rapeseed and palm oils, or the product sold under the name Softisan 100 Cremer by the company Oleo,
partially hydrogenated olive oil, in particular the compound sold under the name Beurrolive by the company Soliance,
hydrogenated sunflower oil,
hydrogenated castor oil,
hydrogenated castor oil derivatives, in particular Thixinr from Rheox,
hydrogenated coconut kernel oil,
hydrogenated palm oil,
hydrogenated coconut oil,
hydrogenated cocoglycerides,
the waxes obtained by hydrogenation of castor oil esterified with cetyl alcohol,
behenyl alcohol,
and/or mixtures thereof.

According to a preferred embodiment, a composition according to the invention comprises at least one wax of plant origin with a melting point above 25° C. chosen from carnauba wax, partially hydrogenated wax esters of jojoba oil, hydrogenated jojoba oil (INCI name: Hydrogenated jojoba oil), fatty acid esters of polyglycerol, hydrogenated cocoglycerides, behenyl alcohol, and mixtures thereof.

More preferentially, a composition according to the invention comprises at least hydrogenated jojoba oil.

According to a particular embodiment, the waxes of plant origin with a melting point above 25° C. according to the invention are present in a content ranging from 0.01% to 20% by weight, in particular from 0.05% to 10% by weight, more particularly from 0.1% to 5% by weight, relative to the total weight of the composition.

According to a preferred embodiment, a composition according to the invention comprises from 0.01% to 10% by weight of hydrogenated jojoba oil, in particular from 0.05% to 8% by weight, more particularly from 0.1% to 5% by weight, and preferably from 0.5% to 3% by weight, relative to the total weight of the composition.

### Oily Phase

The composition according to the invention comprises an oily phase. The proportion of the oily phase of the emulsion may range, for example, from 0.1% to 50% by weight, preferably from 1% to 30% by weight, even more preferentially from 2% to 20% by weight and better still from 4% to 15% by weight.

This indicated amount does not comprise the content of anionic surfactants as defined previously.

For the purposes of the invention, the oily phase includes any fatty substance that is liquid at room temperature and atmospheric pressure, generally oils, or that is solid at room temperature and atmospheric pressure, like waxes, or any pasty compound, which are present in said composition, with the exception of the fatty alcohols as defined previously.

### Aqueous Phase

The composition according to the invention comprises an aqueous phase.

The aqueous phase of the composition in accordance with the invention comprises at least water and the polyol(s) as defined previously. The amount of aqueous phase may range from 50% to 99% by weight, preferably from 60% to 97% by weight and better still from 70% to 95% by weight, relative to the total weight of the composition. According to one embodiment, the amount of aqueous phase may range from 50% to 92% by weight, preferably from 60% to 90% by weight and better still from 70% to 90% by weight, relative to the total weight of the composition. The amount of water may represent all or some of the aqueous phase and it is generally at least 25% by weight relative to the total weight of the composition, preferably at least 35% by weight, better still at least 40% by weight.

The aqueous (or hydrophilic) phase of the composition according to the invention may also contain any water-soluble or water-dispersible additive.

Water-soluble additives that may also be mentioned include primary alcohols, i.e. an alcohol including from 1 to 6 carbon atoms, such as ethanol and isopropanol. It is preferably ethanol. The addition of such an alcohol may notably be suitable when the composition according to the invention is used as a product for the body or the hair.

The amount of water-soluble or water-dispersible additives in the composition of the invention may range, for example, from 0% to 50% by weight, preferably from 0.5% to 30% by weight and even more preferentially from 1% to 20% by weight, relative to the total weight of the composition.

According to a particular embodiment of the invention, the pH of the composition is between 4 and 9 and more preferentially between 4.5 and 7.

### Cosmetic Active Agents and Adjuvants

The composition according to the invention may also comprise at least one additional cosmetic active agent.

In the context of the present invention, the term “additional active agent” means a compound which has, by itself, that is to say not requiring the intervention of an external agent in order to activate it, a biological activity.

The additional active agent(s) may notably be chosen from:
vitamins and derivatives thereof, notably esters thereof, such as tocopherol (vitamin E) and esters thereof (for instance tocopheryl acetate), ascorbic acid (vitamin C) and derivatives thereof, panthenol, niacinamide or vitamin B3;
known anti-ageing active agents, such as hyaluronic acid compounds, and notably sodium hyaluronate, retinol and derivatives thereof, salicylic acid compounds and in particular 5-n-octanoylsalicylic acid (capryloylsalicylic acid), caffeine, adenosine, C-β-D-xylopyranoside-2-hydroxypropane and the sodium salt of (3-hydroxy-2-pentylcyclopentyl)acetic acid;
sunscreens;
menthol;
and mixtures thereof.

The composition according to the invention may also include at least one additive chosen from the usual adjuvants in the cosmetic field, such as preserving agents, fragrances, film-forming polymers, thickening polymers, pH adjusters (acids or bases), preserving agents, fillers, dyes, nacres, pigments and dispersants, and mixtures thereof.

The additional active agent used in the composition according to the invention may represent from 0.0001% to 40%, preferably from 0.01% to 3%, better still from 0.01% to 20% by weight, or from 0.01% to 15% by weight relative to the total weight of the composition. Needless to say, a person skilled in the art will take care to select this or these optional additional compound(s), and/or the amount thereof, such that the advantageous properties of the composition according to the invention are not, or are not substantially, adversely affected by the envisioned addition.

### Cosmetic Composition

The composition used according to the invention may be in any galenic form normally used in the cosmetics field.

The composition according to the invention is in the form of an oil-in-water emulsion. It includes an oily phase dispersed in an aqueous phase.

According to a preferred embodiment, the compositions in accordance with the invention are not solid.

According to another preferred embodiment, the compositions in accordance with the invention are not in stick form.

According to a particularly preferred embodiment, the compositions according to the invention are not solid and are not in stick form.

Advantageously, the compositions according to the invention are in the form of a non-solid galenic, ranging from a serum texture to a creamy texture that can be kept in a jar.

Advantageously, the viscosity of a composition of the invention ranges between 100 cP (centipoises), that is to say 0.1 Pa·s (Pascal·second) (mobile internal measurement 2, 21UD—fluid milk type) and 20 040 cP, that is to say 20.04 Pa·s (mobile internal measurement 4, 85UD—heavy cream type) measured at 25° C.

The viscosity of a composition of the invention can be measured by any method known to the skilled person, and in particular by the following conventional method. For example, measurement can be carried out at 25° C. using a Contraves TV or Rheomat RM180, equipped with a mobile rotating at 200 rpm. The person skilled in the art can choose the mobile used to measure viscosity, for example from M1, M2 or M4 mobiles, on the basis of his general knowledge, so as to be able to carry out the measurement.

In addition, the composition according to the invention may be more or less fluid and may have the appearance of a gel, a white or coloured cream, an ointment, a milk, a lotion, a serum, a paste or a mousse.

More particularly, the composition according to the invention is intended for topical application.

The composition according to the invention may comprise any constituent usually employed in the envisaged topical application and administration.

Preferably, such a composition is not intended to be rinsed off after application.

The composition according to the invention may be intended to be applied to the skin.

Preferably, the skin is the skin of the face and/or of the body, in particular of the face and/or of the neck and/or of the hands, preferably of the face and/or of the neck.

As specified, the composition according to the invention is advantageously in the form of an oil-in-water emulsion.

The composition may alternatively be in the form of a face and/or body care or makeup product, and may be packaged, for example, in the form of a cream in a jar or a fluid in a tube or a pump bottle or a dropper bottle.

The composition according to the invention may be manufactured via any known process generally used in the cosmetics field.

The ingredients are mixed before forming, in the order and under conditions readily determined by a person skilled in the art.

### Uses and Processes

According to one of its aspects, the present invention relates to the use of a composition as described previously in the cosmetic field, and in particular for caring for, protecting and/or making up the skin of the body or face, or for haircare.

According to another of its aspects, the invention relates to a process for the cosmetic treatment of a keratin material in which a composition as defined in the present invention is applied to the keratin material.

The cosmetic uses and processes considered according to the invention are non-therapeutic.

The cosmetic use or process of the invention is performed by topically administering a composition according to the invention.

Topical administration consists of the external application to the skin of cosmetic compositions according to the usual techniques for the use of these compositions.

By way of illustration, the cosmetic use or process according to the invention may be performed by topical application, for example daily, of at least one composition according to the invention, which may be formulated, for example, in the form of a gel, a white or coloured cream, an ointment, a milk, a lotion, a serum, a paste or a foam.

According to one embodiment, the application is repeated, for example, once or twice a day for one or more days and generally over an extended period of at least 4 weeks, or even 4 to 15 weeks, with, where appropriate, one or more periods of stoppage.

According to one embodiment, the application is daily (once a day) and generally over an extended period of at least 4 weeks, or even 4 to 15 weeks, with, where appropriate, one or more periods of stoppage.

According to an embodiment variant, the composition according to the invention will be applied to areas of skin that have been previously cleansed using a washing solution.

Moreover, treatment combinations optionally with topical forms may be envisaged, so as to complement or reinforce the activity of the composition according to the invention.

A topical treatment with the composition according to the invention, combined with an ancillary composition intended for topical application, this ancillary composition possibly being impregnated onto a support such as a wipe, is conceivable.

Thus, the present invention relates in particular to a cosmetic process for treating a keratin material, comprising:
a) a step of topical application to the skin of a washing solution;
b) a step of topical application to the skin of a composition according to the invention; and
c) optionally, a step of topical application of a composition different from the composition applied in step b);
it being possible for steps b) and c) to be performed simultaneously or successively.

Such a composition applied in step c) can be termed a “supplementary composition”.

The washing solution may notably be in various forms, such as a solution, an aqueous solution, a lotion, a milky lotion, a cream, a gel, a liquid gel, a paste, a serum, a suspension, a dispersion, a fluid, a milk, an emulsion (O/W or W/O form), or others, preferably in the form of a solution or an aqueous gel.

Throughout the description, including the claims, the terms “between . . . and . . . ”, and “ranging from . . . to . . . ” should be understood as meaning limits included, unless otherwise specified.

The examples that follow illustrate the present invention without limiting the scope thereof.

### EXAMPLES

### Materials and Methods

### Stability Measurement

The stability of the compositions may be evaluated according to the following protocol.

The properties of the composition to be tested are evaluated immediately after its preparation and 24 hours later.

The composition to be tested is then stored for 2 months at various temperatures: at room temperature or “RT” (about 25° C.), at 4° C. and at 45° C. The temperature may be regulated using an oven, for example the Bio Concept machine from Firlabo.

The properties are evaluated after 1 month and 2 months of storage under the conditions described above.

The properties evaluated are:
the pH of the composition, measured using a pH-meter;
the viscosity of the composition, measured with a viscometer, for example a Rheomat RM 100 viscometer from Lamy Rheology according to the CID-012-02 method;
the appearance of the composition, in particular its microscopic appearance evaluated by observation of the composition under a light microscope between slide and coverslip, at X10 or even X100 magnification;
the colour and odour of the composition; and
Phenomena associated with destabilization of the composition will be observed, such as the appearance of creaming, coalescence, sedimentation, flocculation, the formation of a film on the surface, mottling, etc.

The closer the properties of the composition after 1 month and/or 2 months of storage remain to those initially measured at 24 h at 25° C., the more the composition can be considered as remaining stable.

In particular, if the composition remains smooth and homogeneous, with no signs of destabilization and with no change in colour or odour, then it is considered to be stable.

### Example 1

A composition according to the invention (I1) and a composition outside the invention (C1), in particular comprising a non-silicone oil content of less than 8% by weight (in this case 5% by weight) relative to the total weight of the composition, were prepared.

These compositions, in the form of oil-in-water emulsions, comprise the compounds detailed in Table 1 below, in contents expressed as weight percentages relative to the total weight of the composition.

TABLE 1

| | | | C1 |
|---|---|---|---|
| | | I1 | (outside the |
| Phase | Compound | (invention) | invention) |
| A | Cetearyl alcohol | 6.30 | 6.30 |
| | Preserving agents | 0.50 | 0.50 |
| A | Water | qs 100 | qs 100 |
| A | Chelating agent | 0.25 | 0.25 |
| A | Caprylyl glycol | 0.30 | 0.30 |
| A | Sodium methyl stearoyl taurate | 0.70 | 0.70 |
| | (Nikkol SMT sold by the company Nikko) | | |
| B | Oleyl erucate (Cetiol J 600 sold | 7.50 | 2.50 |
| | by the company BASF) | | |
| | Nacre | 0.14 | 0.14 |
| | C15-19 Alkane (Emogreen L15 sold | 7.50 | 2.50 |
| | by the company SEPPIC) | | |
| C | Glycerol | 20.00 | 20.00 |
| D | Ammonium acryloyldimethyltaurate/ | 0.35 | 0.35 |
| | VP copolymer (Aristoflex AVC sold | | |
| | by the company Clariant) | | |
| E | Water | 0.98 | 0.98 |
| E | Citric acid | 0.02 | 0.02 |
| F | Nacre | 0.35 | 0.35 |

### Preparation Protocol

In a tank, the components of phase A are heated to 75° C. until a homogeneous medium is obtained by paddle stirring without turbomixing. Phase A is turbomixed at 75° C. for 10 minutes (3500 rpm). The components of phase B, heated to 75° C. with magnetic stirring, are added to this heated mixture, while turbomixing for 10 minutes (3500 rpm). Glycerol is added to this mixture and the resulting mixture is cooled to 40° C. with stirring (3500 rpm) and under vacuum. The components of phase D are then added with stirring and under vacuum, followed by the components of the other phases.

Finally, a final homogenization phase is performed down to a temperature of 30° C., with continued stirring and under vacuum, the turbomixing being reduced if necessary.

### Example 2

### Results

The stability and the sensory effect of compositions I1 and C1 were evaluated. The stability results are reported in Table 2 below.

TABLE 2

| | I1 | C1 |
|---|---|---|
| Stability | (invention) | (outside the invention) |
| STABILITY | Appearance/Colour/Odour: | Appearance/Colour/Odour: |
| 24 hours | Supple pink cream | Supple pink cream |
| | Viscosity | Viscosity |
| | Viscosity (Spindle 3) = T30 sec = | Viscosity (Spindle 3) = T30 sec = |
| | 3.16 Pa.s; T10 min = 2.82 Pa.s | 3.04 Pa.s; T10 min = 2.99 Pa.s |
| | pH | pH |
| | pH = 6.3 | pH = 6.4 |
| | Light microscopy: | Light microscopy: |
| | Microscopy (X10): | Microscopy (X10): Fine and tight |
| | LNP Fine and tight emulsion, sharp | emulsion, sharp edges |
| | edges | Anisotropy in polarized light |
| | Anisotropy in polarized light | |
| STABILITY | COMPLIANT | COMPLIANT |
| 1 M 4° C./RT | Appearance/Colour/Odour: | Appearance/Colour/Odour: |
| (25° C.)/45° C. | compliant | compliant |
| | Light microscopy: compliant | Light microscopy: compliant |
| STABILITY | COMPLIANT | NON-COMPLIANT |
| 2 M 4° C./RT | Appearance/Colour/Odour: | Appearance/Colour/Odour: |
| (25° C.)/45° C. | compliant | compliant |
| | Light microscopy: | Light microscopy: |
| | at 4° C. and at RT: compliant | at 4° C. and at RT: compliant |
| | Viscosity: | Viscosity: |
| | Compliant | non-compliant |
| | at RT (25° C.): | at RT (25° C.): |
| | Viscosity (spindle 3): T 30 sec = 2.53 | Viscosity (spindle 3): T 30 sec = 2.20 |
| | Pa.s; T 10 min = 2.66 Pa.s | Pa.s; T 10 min = 2.74 Pa.s |
| | at 45° C. (returned to RT 25° C. before | at 45° C. (returned to RT 25° C. before |
| | measuring the viscosity): | measuring the viscosity): |
| | Viscosity (spindle 3) = T 30 sec = | Viscosity (spindle 3) = T 30 sec = |
| | 2.95 Pa.s; T 10 min = 3.04 Pa.s | 0.90 Pa.s; T 10 min = 2.57 Pa.s |
| | pH | pH |
| | pH at RT (25° C.) = 6.2 | pH at RT (25° C.) = 6.5 |
| | pH at 45° C. (returned to RT 25° C. | pH at 45° C. (returned to RT 25° C. |
| | before measuring the pH) = 6.3 | before measuring the pH) = 6.5 |

In conclusion, only the composition according to the invention I1 is stable after 2 months, whether at 4° C., at room temperature or at 45° C. Specifically, it is compliant in terms of appearance, colour, odour, viscosity and pH.

Composition C1, which is outside the invention, is non-compliant after 2 months in terms of viscosity.

As regards the sensory evaluation, a panel of nine experts took part in this study on the basis of a tactile and visual evaluation. The compositions were applied to the back of the hand.

Composition I1 according to the invention was found to afford good glidance. Moreover, it is described as being sparingly tacky and has little soapiness.

### Example 3

### Preparation of a Composition Outside the Invention

A composition outside the invention (C2) was prepared. This composition, in the form of an oil-in-water emulsion, comprises the compounds detailed in Table 3 below, in contents expressed as weight percentages relative to the total weight of the composition. The composition has a polyol content of less than 15% by weight relative to the total weight of the composition.

TABLE 3

| | | C2 |
|---|---|---|
| | | (outside the |
| Phase | Compound | invention) |
| A | Cetearyl alcohol | 6.30 |
| | Preserving agents | 0.50 |
| A | Water | qs |
| | | 100 |
| A | Chelating agent | 0.25 |
| A | Caprylyl glycol | 0.30 |
| A | Sodium methyl stearoyl taurate | 0.70 |
| | (Nikkol SMT sold by the company Nikko) | |
| B | Oleyl erucate (Cetiol J 600 sold | 7.50 |
| | by the company BASF) | |
| | Nacre | 0.14 |
| | C15-19 Alkane (Emogreen L15 | 7.50 |
| | sold by the company SEPPIC) | |
| C | Glycerol | 12.00 |
| D | Ammonium acryloyldimethyltaurate/ | 0.35 |
| | VP copolymer (Aristoflex AVC | |
| | sold by the company Clariant) | |
| E | Water | 0.98 |
| E | Citric acid | 0.02 |
| F | Nacre | 0.35 |

### Preparation Protocol

The same protocol as that used in Example 1 is used in this example.

### Example 4

### Results

The stability of compositions I1 and C2 was evaluated. The stability results are reported in Table 4 below.

TABLE 4

| | I1 | C2 |
|---|---|---|
| Stability | (invention) | (outside the invention) |
| STABILITY 24 | Appearance/Colour/Odour: | Appearance/Colour/Odour: |
| hours | Supple pink cream | Supple pink cream |
| | Viscosity | Viscosity |
| | Viscosity (Spindle 3) = T30 sec = | Viscosity (Spindle 3) = T30 sec = |
| | 3.16 Pa.s; T10 min = 2.82 Pa.s | 3.07 Pa.s; T10 min = 2.70 Pa.s |
| | pH | pH |
| | pH = 6.3 | pH = 6.4 |
| | Light microscopy: | Light microscopy: |
| | Microscopy (X10): | Microscopy (X10): Fine and tight |
| | LNP Fine and tight emulsion, sharp | emulsion, sharp edges |
| | edges | Anisotropy in polarized light |
| | Anisotropy in polarized light | |
| STABILITY | COMPLIANT | COMPLIANT |
| 1 M 4° C./RT | Appearance/Colour/Odour: | Appearance/Colour/Odour: |
| (25° C.)/45° C. | compliant | compliant |
| | Light microscopy: compliant | Light microscopy: compliant |
| STABILITY | COMPLIANT | NON-COMPLIANT |
| 2 M 4° C./RT | Appearance/Colour/Odour: | Appearance/Colour/Odour: |
| (25° C.)/45° C. | compliant | compliant |
| | Light microscopy: | Light microscopy: |
| | compliant | compliant |
| | Viscosity: | Viscosity: |
| | Compliant | non-compliant |
| | at RT (25° C.): | at RT (25° C.): |
| | Viscosity (spindle 3): T 30 sec = 2.53 | Viscosity (spindle 3): T 30 sec = 0.07 |
| | Pa.s; T 10 min = 2.66 Pa.s | Pa.s; T 10 min = 2.49 Pa.s |
| | at 45° C. (returned to RT 25° C. before | at 45° C. (returned to RT 25° C. before |
| | measuring the viscosity): | measuring the viscosity): |
| | Viscosity (spindle 3) = T 30 sec = | Viscosity (spindle 3) = T 30 sec = |
| | 2.95 Pa.s; T 10 min = 3.04 Pa.s | 2.07 Pa.s; T 10 min = 2.07 Pa.s |
| | pH | pH |
| | pH at RT (25° C.) = 6.2 | pH at RT (25° C.) = 6.6 |
| | pH at 45° C. (returned to RT 25° C. | pH at 45° C. (returned to RT 25° C. |
| | before measuring the pH) = 6.3 | before measuring the pH) = 6.6 |

In conclusion, only the composition according to the invention I1 is stable after 2 months, whether at 4° C., at room temperature or at 45° C. Specifically, it is compliant in terms of appearance, colour, odour, viscosity and pH.

Composition C2, which is outside the invention, is non-compliant after 2 months in terms of viscosity.

### Example 5

### Preparation of the Compositions According to the Invention

Compositions according to the invention (2), (13), (14) and (5) were prepared. These compositions, in the form of oil-in-water emulsions, comprise the compounds detailed in Table 5 below, in contents expressed as weight percentages relative to the total weight of the composition.

TABLE 5

| Phase | Compound | I2 | I3 | I4 | I5 |
|---|---|---|---|---|---|
| A | Water | qs | qs | qs | qs |
| | | 100 | 100 | 100 | 100 |
| A | Cetearyl alcohol | 4.50 | 4.50 | 4.50 | 4.50 |
| | Preserving agent | 0.50 | 0.50 | 0.50 | 0.50 |
| A | Caprylyl glycol | 0.30 | 0.30 | 0.30 | 0.30 |
| | Poly C10-30 alkyl acrylate | — | 0.57 | — | 0.38 |
| A | Chelating agent | 0.25 | 0.25 | 0.25 | 0.25 |
| A | Sodium methyl stearoyl taurate (Nikkol SMT sold by | 0.50 | 0.50 | 0.50 | 0.50 |
| | the company Nikko) | | | | |
| B | Octyldodecanol | — | 1.50 | 1.50 | — |
| B | Oleyl erucate (Cetiol J 600 sold by the company BASF) | 5.00 | 5.00 | 5.00 | 5.00 |
| | Capryloyl salicylic acid | — | 0.15 | 0.15 | — |
| B | Hydrogenated jojoba oil | 1.00 | 1.00 | 1.00 | 1.00 |
| B | C15-19 alkane (Emogreen L15 sold by the company | 5.00 | 5.00 | 5.00 | 5.00 |
| | SEPPIC) | | | | |
| C | Glycerol | 20.00 | 20.00 | 20.00 | 20.00 |
| D | Xanthan gum | — | 0.10 | 0.10 | 0.10 |
| | Sodium hyaluronate | | 0.10 | 0.10 | 0.10 |
| D | Ammonium acryloyldimethyltaurate/VP copolymer | 0.35 | — | 0.50 | — |
| | (Aristoflex AVC sold by the company Clariant) | | | | |
| | Ammonium acryloyldimethyltaurate/Steareth-25 | — | 0.50 | — | 0.50 |
| | methacrylate crosspolymer (Aristoflex HMS sold by the | | | | |
| | company Clariant) | | | | |
| E | Hydroxypropyl tetrahydropyrantriol | — | 4.30 | 4.30 | 4.30 |
| E | Sodium dilauramidoglutamide lysine | — | 0.35 | — | 0.35 |
| E | Citric acid | 0.02 | — | — | 0.02 |
| F | Niacinamide | — | 3.00 | 3.00 | — |
| F | Nacre | — | — | 2.55 | — |
| G | Silica silylate (Dowsil VM-2270 Aerogel Fine Particles | — | 0.14 | 1 | 0.09 |
| | sold by the company Dow Corning) | | | | |
| G | Cellulose (Cellulobeads USF sold by the company Daito | — | 0.70 | — | 0.47 |
| | Kasei Kogyo) | | | | |
| G | Nacres | — | 2 | — | — |
| H | Denatured alcohol | — | 3.00 | — | 3.00 |
| H | Menthol | — | 0.15 | — | 0.15 |

### Preparation Protocol

The same protocol as that used in Example 1 is used in this example.

Compositions (I2) to (I5) have a good level of glidance and are sparingly tacky, while at the same time having little soapiness.

1-18. (canceled)

19. An oil-in-water emulsion composition, comprising:
(a) at least one saturated C16-C22 fatty alcohol;
(b) at least one anionic surfactant comprising at least one C16-C22 hydrocarbon-based chain;
(c) at least one polyol present in an amount greater than 15% by weight, relative to the total weight of the composition, and
(d) at least one non-silicone oil present in an amount of greater than or equal to 8% by weight, relative to the total weight of the composition,
wherein the composition is free of silicone compounds,
wherein an oily phase is dispersed in an aqueous phase in the oil-in-water emulsion composition.

20. The composition of claim 19, wherein the (b) at least one anionic surfactant comprises C16-C22 hydrocarbon-based chain.

21. The composition of claim 19, wherein the (a) at least one saturated C16-C22 fatty alcohol is chosen from cetyl alcohol, hexadecanol, stearyl alcohol, octadecanol, behenyl alcohol, or mixtures thereof.

22. The composition of claim 19, wherein the (a) at least one saturated C16-C22 fatty alcohol is chosen from cetyl alcohol, stearyl alcohol, or mixtures thereof.

23. The composition of claim 19, wherein the (a) at least one saturated C16-C22 fatty alcohol is chosen from a mixture of C16 and C18 fatty alcohols.

24. The composition of claim 19, wherein the total amount of (a) saturated C16-C22 fatty alcohols ranges from 2.5% to 10% by weight, relative to the total weight of the composition.

25. The composition according of claim 19, wherein the (b) at least one anionic surfactant comprising at least one C16-C22 hydrocarbon-based chain is chosen from surfactants comprising at least one sulfonate function, acylglutamates, or mixtures thereof.

26. The composition of claim 19, wherein the (b) at least one anionic surfactant comprising at least one C16-C22 hydrocarbon-based chain is chosen from surfactants of formula (I): 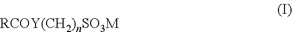
wherein:
R represents a saturated, linear or branched C16-C22 alkyl group;
Y represents —O— or —NR₁— with R₁ representing a linear or branched C1-C3 alkyl group;
M is chosen from groups formed by hydrogen, alkali metals, alkaline-earth metals, ammonium, or organic amines; and
n is an integer ranging from 1 to 3.

27. The composition of claim 26, wherein the (b) at least one anionic surfactant comprising at least one C16-C22 hydrocarbon-based chain is sodium N-stearoyl-N-methyltaurate.

28. The composition of claim 19, wherein (b) anionic surfactants comprising at least one C16-C22 hydrocarbon-based chain are present in a total amount ranging from 0.05% to 5% by weight, relative to the total weight of the composition.

29. The composition of claim 19, wherein the weight ratio of the total amount of saturated C16-C22 fatty alcohols (a) to the total amount of anionic surfactants comprising at least one C16-C22 hydrocarbon-based chain (b) ranges from 10:1 to 3:2.

30. The composition of claim 19, wherein the (c) at least one polyol comprises carbon atoms in a number ranging from 2 to 20.

31. The composition of claim 19, wherein the (c) at least one polyol comprises from 2 to 12 hydroxyl functions.

32. The composition of claim 19, wherein the polyols (c) are present in a total amount ranging from 15.1% to 50% by weight, relative to the total weight of the composition.

33. The composition of claim 19, wherein the (d) at least one non-silicone oil is chosen from: branched or linear alkanes comprising more than 8 carbon atoms; hydrocarbon-based oils of animal origin; linear or branched hydrocarbons of mineral or synthetic origin; hydrocarbon-based oils of plant origin; synthetic ethers comprising from 10 to 40 carbon atoms; synthetic esters of formula R₁COOR₂, wherein R₁ represents a residue of a linear or branched fatty acid comprising from 1 to 40 carbon atoms, R₂ represents a hydrocarbon-based chain, with the proviso that R₁+R₂ is an integer greater than or equal to 10; polyol esters and pentaerythritol esters; diol dimer and diacid dimer esters; fatty amides; fatty alcohols that are liquid at room temperature, with a branched carbon-based chain comprising from 12 to 26 carbon atoms; C19-C22 fatty acids; carbonates; or mixtures thereof.

34. The composition of claim 19, wherein the non-silicone oils (d) are present in a total amount of franging from 8% to 40% by weight, relative to the total weight of the composition.

35. A cosmetic method for treating keratin material, comprising applying to the keratin material a composition comprising:
(a) at least one saturated C16-C22 fatty alcohol;
(b) at least one anionic surfactant comprising at least one C16-C22 hydrocarbon-based chain;
(c) at least one polyol present in an amount greater than 15% by weight, relative to the total weight of the composition, and
(d) at least one non-silicone oil present in an amount of greater than or equal to 8% by weight, relative to the total weight of the composition,
wherein the composition is free of silicone compounds,
wherein the composition is in the form of an oil-in-water emulsion with an oily phase dispersed in an aqueous phase.
